(19) United States
(12) Patent Application Publication
(10) Pub. No.: US 2026/0335096 A1
(43) Pub. Date: Oct. 29, 2026

(54) Snap-Off Hammertoe Device

(71) Applicants: Erin Lansdale, San Antonio, TX (US); Thomas Zink, San Antonio, TX (US)

(72) Inventors: Erin Lansdale, San Antonio, TX (US); Thomas Zink, San Antonio, TX (US)

(21) Appl. No.: 19/191,730

(22) Filed: Apr. 28, 2025

(51) Int. Cl. A61F 2/42; A61B 17/16; A61F 2/30; A61F 2/46; B33Y 80/00

(52) U.S. Cl. CPC A61F 2/4225; A61B 17/1682; A61F 2/30771; A61F 2/4606; B33Y 80/00; A61F 2002/30561; A61F 2002/30593; A61F 2002/30622; A61F 2002/30851; A61F 2002/3093

(57) ABSTRACT

A snap-off hammertoe device is disclosed, comprising a body component engaged with a shaft extension. The body component comprises a first end which engages the shaft extension and a second end with a penetrating tip. The shaft extension is a snap-off barrel component which is used to insert the hammertoe device and is broken off by means of torsion or bending torque after the body component is inserted. The snap-off barrel component or shaft extension and the body are both cannulated, such that a guidewire can be inserted. Further, the snap-off barrel component or shaft extension and the body are additively manufactured and grown as a single part.

### FIELD OF THE INVENTION

The present invention relates generally to a medical device for use in the field of podiatry. More particularly, the present invention relates to a snap-off cannulated intramedullary fusion implant with a thread feature for self-drilling and self-tapping and a shaft extension. Accordingly, the present disclosure makes specific reference thereto. Nonetheless, it is to be appreciated that aspects of the present invention are also equally applicable to other like applications, devices and methods of manufacture.

### BACKGROUND

A hammertoe is a deformity of the proximal inter-phalangeal joint of the second, third, fourth, or fifth toe causing the toe to be permanently bent and giving the toe the resemblance of a hammer. Initially, hammertoes are flexible and may be corrected with simple measures but, if left untreated, hammertoes may require surgical intervention for correction. Persons with a hammertoe may also have corns or calluses on the top of the middle joint of the toe or on the tip of the toe and may feel pain in their toes or feet and have difficulty finding comfortable shoes.

Hammertoe deformities are commonly corrected with fusion via inserting screws, wires or other similar implants in toes to straighten them. Traditional surgical methods generally include the use of Kirschner wires (K-wires). K-wires require pins protruding through the end of respective toes due to their temporary nature. As a result, K-wires often lead to pin tract infections, loss of fixation, and limitations to a fixed angle. Additional disadvantages of K-wires include migration and breakage of the K-wires thus resulting in multiple surgeries. Further, the limitations of a fixed angle provide for very few options for variability of the angle. If the surgeon chooses an implant with an angle, the orientation of the implant at insertion is crucial to the correct fixation. Due to the various disadvantages of using K-wires, compression screws or devices are being employed as an implant alternative.

Compression screws or devices may provide a more permanent solution than K-wires as such implants do not need removal and have no protruding ends. Further, with the use of compression screws or devices, a patient may wear normal footwear shortly after the respective surgery. There are generally two types of known compression device implants: single-unit implants, which comprise a completely threaded body and do not provide a flexibility to the respective toe in its movement, and two-unit implants, which typically have one unit that is anchored into the proximal phalanx, a second unit that is anchored into the distal phalanx, and a fitting by which the two units are coupled. Either or both of the two units may be threaded or have other anchoring structures such as barbs or splaying arms.

Thus, there exists a need for an improved hammertoe compression device that corrects a hammertoe deformity and provides stable fixation between separate bone fragments. The present invention discloses a snap-off hammertoe device that is a cannulated intramedullary fusion implant with a threaded feature for self-drilling and self-tapping, and a shaft extension. Further, the snap-off hammertoe device is cannulated and can be inserted in conjunction with a guidewire to allow for easier and faster insertion. The cannulation allows for guided insertion and decreases procedure difficulty while maintaining the benefits of using a snap-off hammertoe device. Further, the snap-off hammertoe device comprises a body component comprising insertion features to be used in case of a premature breaking of the barrel component, extraction, or device stripping. Additionally, having the snap-off hammertoe device additively manufactured as one part allows for customizable and variable surface roughness, thread profile and/or diameter, shank diameter, and snap-off methods. Thread modification provides bone fixation with increased rotational fixation stability, pullout-strength, and compression.

The subject matter disclosed and claimed herein, in one embodiment thereof, comprises a snap-off hammertoe device. The device is designed to generate compression and provide stable fixation between separate bone fragments. The device comprises a body and a break-off barrel. The break-off barrel is used to insert the body and is broken off by means of torsion or bending torque after the body is inserted. The device can be cannulated and inserted in conjunction with a guidewire. The body portion of the device can feature insertion features to be used in case of a premature breaking of the barrel or extraction. Additive manufacturing allows for customizable and variable surface roughness, thread profile/diameter, break-off method, and shank diameter. Once the break-off barrel is removed, the remainder of the implant is press-fitted into a pre-drilled hole on the opposing bone.

In this manner, the snap-off hammertoe device of the present invention accomplishes all of the forgoing objectives and provides users with a device that generates compression and provides stable fixation between separate bone fragments. The snap-off hammertoe device provides a threaded feature for self-drilling and self-tapping, and a shaft extension. The shaft extension is used for implantation and is snapped and removed once the device is inserted into bone.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one aspect thereof, comprises a snap-off hammertoe device comprising a body component engaged with a shaft extension. The body component comprises a first end which engages the shaft extension and a second end with a penetrating tip. The shaft extension is a snap-off barrel component which is used to insert the hammertoe device. The body component further comprises an exterior thread on an exterior surface portion of the body component. The snap-off barrel component or shaft extension and the body are both cannulated, such that a guidewire can be inserted. Further, the snap-off barrel component or shaft extension and the body are additively manufactured and grown as a single part.

In one embodiment, the body component comprises an osteo-integrated component which allows bone to grow into it, to help fuse the joint. The osteo-integrated component can be any suitable shape and size as is known in the art. Typically, the osteo-integrated component is round and secured to a middle of the body component or is grown on the body component via additively manufacturing. More than one osteo-integrated component can be utilized, as is known in the art and depending on the needs and/or wants of a user.

In an alternative embodiment, the body component of the snap-off hammertoe device comprises thread ramps and thread patterns that provide rotational stability, increase pullout strength, and prevent screw back-out. Specifically, the thread diameter, profile, and pitch can change across the thread path to provide custom pressure mappings and can be varied along the length of the device to target cortical or cancellous bone purchase at select regions. Further, the shank diameter and thread profile can be increased to increase fixation stability. Specifically, additive manufacturing allows for customizable and variable surface roughness, thread profile and diameter, shank diameter, and snap-off methods.

In another embodiment, the body component comprises insertion features near the first end (proximal end). The insertion features can be utilized in case of a premature breaking of the barrel component or shaft extension, or during extraction. The insertion features provide more solutions for device stripping, as well. Typically, the insertion features are rectangular slots or notches, but can be any suitable shape as is known in the art, such as V-shaped, etc.

In another embodiment, the snap-off hammertoe device is manufactured as an additively printed titanium component. Nonetheless, the snap-off hammertoe device can also be manufactured as a machined titanium alloy, stainless steel, or any other suitable biocompatible material component as is known in the art. Further, the osteo-integrated component of the device can include an inner mesh structure, which is generated by 3D printed PEEK, titanium, or any other suitable biocompatible material as is known in the art, and which acts as a scaffold for bone.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIGS. 1A-1C illustrate multiple views of the snap-off hammertoe device of the present invention in accordance with the disclosed architecture;

FIG. 1D illustrates a top perspective view of the distal end of the snap-off hammertoe device of the present invention in accordance with the disclosed architecture;

FIG. 1E illustrates a top perspective view of the proximal end of the snap-off hammertoe device of the present invention in accordance with the disclosed architecture; and

FIGS. 2A-2D illustrate multiple views of the instruments for inserting and removing the snap-off hammertoe device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

In this application, the words proximal, distal, anterior or plantar, posterior or dorsal, medial and lateral are defined by their standard usage for indicating a particular part or portion of a bone, or directional terms of reference, according to the relative disposition of the natural bone. For example, “proximal” means the portion of a bone nearest the torso, while “distal” indicates the portion of the bone farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body. Further, specifically in regards to the foot, the term “dorsal” refers to the top of the foot and the term “plantar” refers the bottom of the foot.

Similarly, positions or directions may be used herein with reference to anatomical structures or surfaces. For example, as the current devices, instrumentation and methods are described herein with reference to use with the bones of the foot, the bones of the foot, ankle and lower leg may be used to describe the surfaces, positions, directions or orientations of the devices, instrumentation and methods. Further, the devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices, instrumentation and methods, and the aspects, components, features and the like thereof, described herein with respect to the right foot may be mirrored so that they likewise function with the left foot. Further, the devices, instrumentation and methods, and the aspects, components, features and the like thereof, disclosed herein are described with respect to the foot for brevity purposes, but it should be understood that the devices, instrumentation, and methods may be used with other bones of the body having similar structures, for example the upper extremity, and more specifically, with the bones of the wrist, hand, and arm.

As noted above, there is a long felt need in the art for devices and instrumentation that generate compression and provide stable fixation between separate bone fragments. Specifically, devices which are an additively manufactured, cannulated, intramedullary snap-off implant, wherein the snap feature will break with bending torque or high enough torsion loads.

The present invention, in one exemplary embodiment, is a novel snap-off hammertoe device. The device comprises a body component engaged with a shaft extension. The body component comprises a first end which engages the shaft extension and a second end with a penetrating tip. The shaft extension is a snap-off barrel component which is used to insert the hammertoe device. The body component further comprises an exterior thread on an exterior surface portion of the body component. Further, the snap-off barrel component or shaft extension and the body are additively manufactured and grown as a single part.

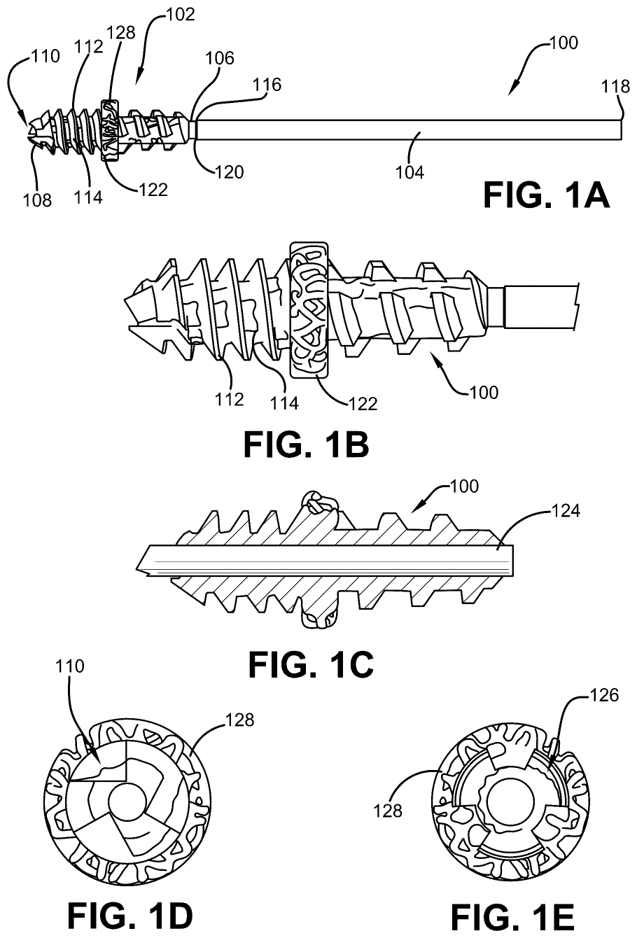

Referring initially to the drawings, FIGS. 1A-C illustrate multiple views of the snap-off hammertoe device 100 of the present invention. In the present embodiment, the snap-off hammertoe device 100 is an improved hammertoe device that generates compression and provides stable fixation between separate bone fragments. The device 100 is especially designed to allow any doctor, surgeon, etc., or any other suitable user as is known in the art, to insert the implant easier and faster because of its insertion compatibility with a wire driver and its break-off feature. Further, the cannulation allows for guided insertion and allows for K-wires to be left in place for additional fusion points. More specifically, the device 100 comprises a body component 102 engaged with a shaft extension 104. The shaft extension 104 is used to insert the hammertoe device 100 and is broken off by means of torsion or bending torque after the hammertoe device 100 is inserted.

The body component 102 comprises a first end (proximal end) 106 which engages the shaft extension 104 and a second end (distal end) 108 with a penetrating tip 110. The shaft extension 104 is a snap-off barrel component which is used to insert the hammertoe device 100. The body component 102 further comprises an exterior thread 112 on an exterior surface portion of 114 the body component 102.

As shown in FIG. 1D, the penetrating tip 110 and exterior thread 112 are configured to advance the snap-off hammertoe device 100 into the bone once inserted. However, the snap-off hammertoe device 100 also comprises a snap-off barrel component or shaft extension 104 which is used to insert the hammertoe device 100. The snap-off barrel component 104 engages and extends proximally from the body component 102. The snap-off barrel component 104 is cylindrical in shape and comprises a first end 116 engaged with the proximal end 106 of the body component 102 and a second end 118 configured for engagement by a pin driver, hand driver, spine retention driver, or other suitable inserter (all not shown). Although dimensions of the snap-off barrel component 104 (i.e., length, width, and height) are important design parameters for good performance, the snap-off barrel component 104 may be any shape or size that ensures optimal performance during use.

In use, the snap-off barrel component 104 is inserted into the collet of the pin driver or other inserter, a user depresses a lever to engage the clamping mechanism which then secures the snap-off barrel component 104 within the pin driver or other inserter. Once secured, the pin driver or other inserter is used to rotate the snap-off barrel component 104 and thus drive the snap-off hammertoe device 100 into bone. Once the snap-off hammertoe device 100 is inserted in vivo, the pin driver or other inserter is removed and the snap-off barrel component 104 is snapped-off of the body component 102. The snap-off barrel component 104 is broken off my means of torsion or bending torque as the hammertoe device 100 is being inserted.

Furthermore, the snap-off barrel component 104 and the body component 102 are additively manufactured and grown as a single part. Specifically, the snap-off hammertoe device 100 is additively manufactured from titanium alloys, magnesium zinc alloys, stainless steel, PEEK (Polyether ether ketone) or other suitable materials as is known in the art. Additively manufacturing the snap-off hammertoe device 100 allows a user to ensure break-off with torque of the snap-off barrel component 104 exactly at the break-off point 120. Specifically, a user would orient the layers of the 3D print in the same direction as the snap-off barrel component 104 would break-off, creating a layer right at the break-off point 120. This ensures break-off with torque at the break-off point 120 and ensures that no fragments of the snap-off barrel component 104 remain in the body component 102 of the hammertoe device 100.

As shown in FIGS. 1A-B, the body component 102 comprises an osteo-integrated component 122 which allows bone to grow into it, to help fuse the joint. The osteo-integrated component 122 can be any suitable shape and size as is known in the art. Typically, the osteo-integrated component 122 is round and secured to a middle of the body component 102 or is grown on the body component 102 via additively manufacturing. More than one osteo-integrated component 122 can be utilized, as is known in the art and depending on the needs and/or wants of a user. Further, the osteo-integrated component 122 can include an inner mesh structure 128, or any other suitable structure as is known in the art, which is generated by 3D printed PEEK, titanium, or any other suitable biocompatible material as is known in the art, and which acts as a scaffold for bone.

In one embodiment, the osteo-integrated component 122 can act as a stop, preventing the user from inserting the device 100 too deep, or too far distal. Specifically, once the osteo-integrated component 122 hits the face of the intermedial phalanx, the user is prevented from going any farther.

As shown in FIG. 1C, the snap-off barrel component 104 and the body component 102 can all be cannulated 124 such that the snap-off hammertoe device 100 is inserted in conjunction with a guidewire (not shown). Thus, the cannulation 124 allows for guided insertion of the hammertoe device 100 and decreases procedure difficulty while maintaining the benefits of using a snap-off hammertoe device 100. Specifically, a guidewire is inserted into the collet of the pin driver, a user depresses a lever to engage the clamping mechanism which then secures the guidewire within the pin driver. Once secured, the pin driver is used to rotate the guidewire and drive it into bone. Once the guidewire is in place, the pin driver is removed.

As shown in FIG. 1E, the body component 102 of the snap-off hammertoe device 100 comprises an insertion feature 126. Specifically, the proximal end 106 of the body component 102 can possess insertion features depending on the wants and/or needs of a user. The insertion features 126 are a plurality of notches which are positioned tangentially down the proximal end 106 of the body component 102 for receipt of a portion of a driver or insertion device (200). Specifically, the plurality of notches 126 engage with a pronged inserter 200. The notches 126 allow a user to still be able to engage the body component 102 even if the snap-off barrel component 104 is not broken-off at exactly the break-off point 120 causing fragments to remain in the body component 102, obscuring the insertion features 126. Any number of notches 126 can be grown on the body component 102 during the additively manufacturing process depending on the needs and/or wants of a user.

Furthermore, the body component 102 of the snap-off hammertoe device 100 comprises thread patterns that provide rotational stability, increase pullout strength, and prevent screw back-out. Further, the thread diameter, profile, and pitch can change across the thread path to provide custom pressure mappings and can be varied along the length of the body component 102 to target cortical or cancellous bone purchase at select regions. Further, the shank diameter and thread profile can be increased to increase fixation stability. Specifically, the additive manufacturing process allows for customizable and variable surface roughness, thread profile and diameter, shank diameter, and snap-off methods.

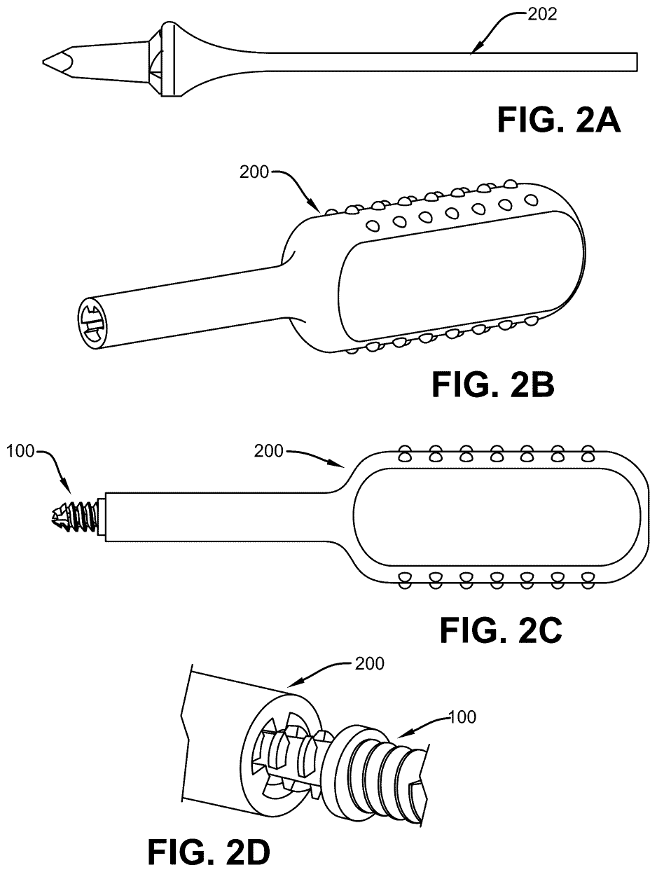

As shown in FIG. 2A, the drill 202 is used to drill a hole in the proximal bone. As shown in FIGS. 2B-D, the insertion device 200 may then be used in the instance that the shaft extension or snap-off barrel component 104 is broken-off prematurely. Other suitable instruments as is known in the art can be utilized to accommodate varying anatomies, purposes, and desired outcomes.

A surgical method for implanting the snap-off hammertoe device 100 into a joint space, will now be described. The method utilizes some of the devices, instruments, features, aspects, components and the like described above, and therefore reference will be made to the above described embodiments, such as the illustrated embodiments presented in the figures and discussed above. However, such references are made for exemplary purposes only and are not intended to limit the surgical method beyond the specifically recited steps. Further, the surgical method may be discussed under the umbrella of particular bones, but such an application is not intended to be limiting and the method described herein may be used or conducted with bone or other tissue not specifically discussed herein without departing from the spirit and scope of the surgical method.

Create a dorsal incision over the dorsal side, over the medial phalanx and proximal phalanx joint to expose the joint. Retract soft tissue as necessary and remove residual cartilage.

Drill the proximal phalanx, and then thread the device 100 into the intermediate phalanx. Once in the intermediate phalanx, a user snaps the snap-off barrel component 104 off of the device 100 leaving a protruding section from the body component 102. Then, the protruding section of the body component 102 is press fit into the drill hole in the proximal phalanx.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “snap-off hammertoe device”, “hammertoe device”, and “device” are interchangeable and refer to the snap-off hammertoe device 100 of the present invention.

Notwithstanding the forgoing, the snap-off hammertoe device 100 of the present invention can be of any suitable size and configuration as is known in the art without affecting the overall concept of the invention, provided that it accomplishes the above stated objectives. One of ordinary skill in the art will appreciate that the snap-off hammertoe device 100 as shown in FIGS. 1-2 is for illustrative purposes only, and that many other sizes and shapes of the snap-off hammertoe device 100 are well within the scope of the present disclosure. Although the dimensions of the snap-off hammertoe device 100 are important design parameters for user convenience, the snap-off hammertoe device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A snap-off hammertoe device that generates compression and provides stable fixation between separate bone fragments, the snap-off hammertoe device comprising:
a body component; and
a shaft extension, wherein the shaft extension is used to insert the body component into a patient and is broken off by means of torsion or bending torque after the body component is inserted into the patient.

2. The snap-off hammertoe device of claim 1, wherein the body component comprises a first end which engages the shaft extension and a second end having a penetrating tip.

3. The snap-off hammertoe device of claim 2, wherein the shaft extension is a snap-off barrel component which is used to insert the body component.

4. The snap-off hammertoe device of claim 3, wherein the body component further comprises an exterior thread on an exterior surface portion of the body component.

5. The snap-off hammertoe device of claim 4, wherein the body component comprises a penetrating tip, which combines with the exterior thread to advance the body component into a bone of the patient once inserted.

6. The snap-off hammertoe device of claim 5, wherein the snap-off barrel component engages and extends proximally from the body component.

7. The snap-off hammertoe device of claim 6, wherein the snap-off barrel component is cylindrical in shape and comprises a first end engaged with the first end of the body component and a second end configured for engagement by an inserter.

8. The snap-off hammertoe device of claim 7, wherein the snap-off barrel component and the body component are additively manufactured and grown as a single part.

9. The snap-off hammertoe device of claim 8, wherein the snap-off hammertoe device is additively manufactured from a titanium alloy, a magnesium zinc alloy, a stainless steel or a PEEK (Polyether ether ketone).

10. The snap-off hammertoe device of claim 9, wherein additively manufacturing the snap-off hammertoe device allows a user to ensure break-off with torque of the snap-off barrel component exactly at a break-off point.

11. The snap-off hammertoe device of claim 10, wherein the user would orient layers of a 3D print in a same direction as the snap-off barrel component would break-off, thereby creating a layer right at the break-off point, which ensures break-off with torque at the break-off point and ensures that no fragments of the snap-off barrel component remain in the body component.

12. The snap-off hammertoe device of claim 1, wherein the body component comprises an osteo-integrated component which allows bone to grow into it, to help fuse a joint.

13. A snap-off hammertoe device that generates compression and provides stable fixation between separate bone fragments, the snap-off hammertoe device comprising:
a body component comprising a first end which engages a shaft extension and a second end with a penetrating tip; and
a shaft extension which is a snap-off barrel component used to insert the body component into a bone of a patient;
wherein the shaft extension is used to insert the body component and is broken off by means of torsion or bending torque after the body component is inserted into the bone;
wherein the body component comprises a penetrating tip, which combines with an exterior thread on an exterior surface portion of the body component to advance the body component into the bone once inserted;
wherein the snap-off barrel component engages and extends proximally from the body component;
wherein the snap-off barrel component is cylindrical in shape and comprises a first end engaged with the first end of the body component and a second end configured for engagement by an inserter;
wherein a user would orient a plurality of layers of a 3D print in a same direction as the snap-off barrel component would break-off, thereby creating a layer right at a break-off point, which ensures break-off with torque at the break-off point and ensures that no fragments of the snap-off barrel component remain in the body component; and
further wherein the body component comprises an osteo-integrated component which allows bone to grow into it, to help fuse a joint.

14. The snap-off hammertoe device of claim 13, wherein the osteo-integrated component is round and secured to a middle of the body component or is grown on the body component via additively manufacturing.

15. The snap-off hammertoe device of claim 14, wherein multiple osteo-integrated components can be utilized.

16. The snap-off hammertoe device of claim 15, wherein the osteo-integrated component comprises an inner mesh structure, which is generated by 3D printed PEEK or a titanium and which acts as a scaffold for the bone.

17. The snap-off hammertoe device of claim 16, wherein the osteo-integrated component acts as a stop and once the osteo-integrated component hits a face of an intermedial phalanx, the user is prevented from going any farther.

18. The snap-off hammertoe device of claim 13, wherein the snap-off barrel component and the body component are both cannulated, such that the snap-off hammertoe device is inserted in conjunction with a guidewire.

19. The snap-off hammertoe device of claim 13, wherein the body component comprises an insertion feature, which are a plurality of notches positioned tangentially down a proximal end of the body component for receipt of a portion of an insertion device.

20. A method for implanting the snap-off hammertoe device into a joint space, the method comprising the following steps:
providing a snap-off hammertoe device comprising a body component with a snap-off barrel component;
creating a dorsal incision over a dorsal side, over a medial phalanx and a proximal phalanx joint to expose a joint;
drilling an opening in the proximal phalanx, and then threading the snap-off hammertoe device into an intermediate phalanx;
once in the intermediate phalanx, a user snaps the snap-off barrel component off of the snap-off hammertoe device to leaving a protruding section from the body component; and
press fitting the body component into the opening drilled in the proximal phalanx.
